(19) United States
(12) Patent Application Publication
(10) Pub. No.: US 2018/0214846 A1
(43) Pub. Date: Aug. 2, 2018

(54) MATRIX MATERIALS HAVING MECHANICALLY-SHAPED SPONGE ARCHITECTURE AND METHODS OF PREPARING THEREOF

(71) Applicant: Slendine AG, Mezzovico-Vira (CH)

(72) Inventor: Paolo Costa, Tiburon, CA (US)

(21) Appl. No.: 15/884,046

(22) Filed: Jan. 30, 2018

Related U.S. Application Data
(60) Provisional application No. 62/451,990, filed on Jan. 30, 2017.

(51) Int. Cl. B01J 20/28; B01J 20/30; B29C 39/14; B29C 39/26; B29C 44/02; B29C 44/34; B29C 44/56; B29C 44/58; A61F 5/00

(52) U.S. Cl. CPC B01J 20/28045; B01J 20/28047; B01J 20/3007; B01J 20/3035; B29C 39/14; B29C 2793/0045; B29C 44/02; B29C 44/3403; B29C 44/5627; B29C 44/58; A61F 5/0076; B29C 39/26

(57) ABSTRACT

Methods of preparing and using compositions and devices comprising compressible absorbent matrix material having mechanically-shaped sponge architecture are disclosed.

### CROSS REFERENCE TO RELATED APPLICATIONS

This application claims the benefit of U.S. Provisional Patent Application No. 62/451,990 filed Jan. 30, 2017, the entire disclosure of which is incorporated herein by reference.

### FIELD OF THE INVENTION

The present disclosure provides compositions and devices comprising compressible absorbent matrix material having mechanically-shaped sponge architecture, and methods of preparing and using the same.

### BACKGROUND OF THE INVENTION

Current methods of preparing compressible absorbent matrix material are inadequate for producing absorbent material and devices with controlled behavior. For instance, absorbent matrix material produced using currently available methods cannot generate matrix material having controlled and customizable absorptive and compressibility parameters. This is because the methods currently used do not provide adequate control over the architecture of the matrix material, such as the size, shape, and extent of porosity of the matrix material. Therefore, there is a need in the art for new methods of preparing compressible absorbent matrix materials having controlled and customizable absorptive and compressibility parameters.

### SUMMARY OF THE INVENTION

In one aspect, the present disclosure provides a method of preparing an absorbent matrix material having a mechanically-shaped sponge architecture. The method comprises providing a substrate and forming the sponge architecture in the substrate; forming a substrate around a removable porogenic mold substantially having the shape of the sponge architecture and removing the porogenic mold; or combinations thereof. The absorbent matrix material may be compressible. The substrate may be a naturally occurring material or an artificially prepared material. The substrate may also be a solid material, a hydrogel, or a sponge material.

The method may comprise forming the sponge architecture in a substrate. When the method comprises forming the sponge architecture in a substrate, forming the sponge architecture may be selected from the group consisting of drilling the substrate, puncturing the substrate, and combinations thereof. Further, the substrate may be selected from a hydrated substrate, a dry substrate, a partially hydrated substrate, or a combination thereof. The substrate may also be a dry hydratable carbohydrate. The substrate may be a dry hydratable substrate and a dry absorbent matrix material is formed in the dry hydratable substrate. When the substrate may be a dry hydratable substrate and a dry absorbent matrix material is formed in the dry hydratable substrate, the method may further comprise hydrating the dry absorbent matrix material to generate a hydrated absorbent matrix material. When the method comprises forming the sponge architecture in a substrate, the substrate may be a dry hydratable substrate and the method may further comprise hydrating the substrate to generate a hydrated substrate, and forming the sponge architecture in the hydrated substrate.

The method may also comprise forming a substrate around a removable porogenic mold substantially having the shape of the sponge architecture and removing the porogenic mold. For instance, the substrate may be formed around the porogenic mold by growing, building, depositing, or developing the substrate around the porogenic mold. The substrate may be selected from a hydrated substrate, a dry substrate, a partially hydrated substrate, or a combination thereof, or the substrate may be a dry hydratable carbohydrate. The substrate may be a dry hydratable substrate, and the method may further comprise depositing the dry hydratable substrate in the removable mold and hydrating the hydratable substrate to expand and form a hydrated substrate in the mold. The porogenic mold may be removed by detaching the mold from the formed substrate, by eliminating the mold from the formed substrate, or by chemically modifying the porogenic mold to eliminate the mold.

When the matrix material is compressible, the matrix material may have a defined size, and the method may further comprise compressing the absorbent matrix material to reduce the size of the matrix material. The size of the matrix material may be reduced to a size that is less than 50% of an expanded size of the matrix material.

The absorbent matrix material may be a hydrated absorbent matrix material, and the method may further comprise drying the matrix material to form a dried absorbent matrix material. The absorbent matrix material may also be a hydrated absorbent matrix material having a defined size, and the method may further comprise drying the matrix material to form a dried absorbent matrix material and compressing the dried matrix material to reduce the size of the dried matrix material.

In another aspect, the present disclosure provides a compressible absorbent matrix material having mechanically-shaped sponge architecture prepared using a method as described above. The matrix material may be reduced in size and/or volume to a size and/or volume that is less than 50% of an expanded size of the matrix material. One or more member of the compressible absorbent matrix material may be packed into a delivery device.

In another aspect, the present disclosure provides a delivery device comprising one or more members of a compressible absorbent matrix material, the matrix material having a mechanically-shaped sponge architecture prepared using a method as described above.

### BRIEF DESCRIPTION OF THE FIGURES

FIG. 1 depicts one embodiment of a method of preparing a compressible absorbent matrix material having a mechanically-shaped sponge architecture. In the instant embodiment, a dry substrate (panel 1) is provided. The dry substrate is partially or fully hydrated (panel 2) before drilling or puncturing the substrate (panel 3) to form an absorbent matrix material. The resulting matrix material may further be dried (panel 4) and compressed (panel 5).

FIG. 2 depicts one embodiment of a method of preparing a compressible absorbent matrix material having a mechanically-shaped sponge architecture. In the instant embodiment, a dry substrate (dry hydrogel) is introduced into a removable porogenic mold (panel 1; here represented by a mold having a top face and a bottom face) before mating the top and bottom faces of the mold, and hydrating the dry substrate in the mold to expand and form a hydrogel around the mold (panel 2). The mold is then separated from the hydrated substrate, thereby forming the sculpted hydrogel matrix material (panel 3).

### DETAILED DESCRIPTION

New methods of preparing absorbent matrix materials having mechanically-shaped sponge architecture have been invented. As used herein, the term “sponge architecture” is used to describe the design, shape, size, and distribution of pores, cells, and conduits in an absorbent matrix in relation to other pores, cells, and conduits in the matrix material and in relation to the matrix material. As used herein, the term “mechanically-shaped sponge architecture” refers to a precisely defined sponge architecture prepared using methods capable of forming the sponge architecture.

Importantly, having a mechanically-shaped sponge architecture provides for greater control over the sponge architecture of the matrix material of the instant disclosure. For instance, the size, shape, and extent of porosity can be more finely tuned in matrix material having mechanically-shaped sponge architecture when compared to currently available sponge matrices having a sponge architecture that is not mechanically-shaped. Additionally, mechanically-shaped sponge architecture allows for more efficient production of porous sponge matrices with precisely controlled porosity profiles at a significantly cheaper price than other methods currently available. Therefore, using methods of the instant disclosure, compressible absorbent matrix material can be produced having controlled and customizable absorptive and compressibility parameters at a significantly reduced price when compared to other methods of producing compressible absorbent matrix materials. Pore size distribution and the expansion capability of matrix material produced using the instant methods can be greatly improved, when compared to compressible absorbent matrix materials produced using other methods. For instance, matrix material having up to 99% porosity can be produced using the instant methods, thereby greatly enhancing the expansion capability, and ensures an expansion capability of 100 times or more of the compressed volume. The matrix material and the methods of preparing the matrix material are described below.

### I. Absorbent Matrix Material

In one aspect, the present disclosure encompasses an absorbent matrix material. The matrix material has mechanically-shaped sponge architecture. The mechanically-shaped sponge architecture of the matrix material has a defined shape, size, and distribution of pores, cells, and conduits in the matrix material in relation to the matrix material and to other pores, cells, and conduits in the matrix material. The matrix material generally has an internal porosity like a sponge. The matrix material may also be compressible.

The sponge architecture of an absorbent matrix material of the instant disclosure can and will vary, and may be any sponge architecture having a design suitable for the intended use of the matrix material. For instance, a sponge architecture of a matrix material may comprise ordered regular arrays of pores distributed evenly throughout a matrix material. Alternatively, a sponge architecture of a matrix material may be disordered and irregularly distributed in a matrix material. Similarly, a sponge architecture of a matrix material may comprise similarly sized pores throughout a matrix material. Alternatively, a sponge architecture of a matrix material may comprise pores of varying sizes throughout a matrix material. The pores or cells of the sponge-like matrix material may be random and/or amorphous (e.g., as in typical commercially available artificial or natural sponges). Alternatively, in some embodiments, the cells of the sponge material may be more regular and/or geometrically ordered, like a honeycomb or other geometric and/or volumetric arrangement. An individual of skill in the art will recognize that any combination of ordering and sizing of pores in a matrix material may be envisioned.

The mechanically-shaped sponge architecture of the matrix material may also provide a magnifying and/or multiplicative effect on the expansion of the matrix material and its fluid absorption and/or retention capacity, relative to matrix material comprising amorphous and/or non-mechanically-shaped sponge architecture. This magnifying and/or multiplicative effect can be at least 2 times, at least 5 times, at least 10 times, at least 50 times, or at least 100 times, relative to matrix material comprising amorphous and/or non-mechanically-shaped sponge architecture. Accordingly, a much smaller quantity, mass, volume, dosage or amount of the present matrix material(s) of the disclosure is required in these embodiments.

In addition to the mechanically-shaped sponge architecture, a matrix material may further comprise a sponge architecture that was not formed using the mechanical methods of forming the mechanically-shaped sponge architecture described herein. For instance, an absorbent matrix material may comprise a sponge architecture from naturally-formed sponge material or from artificially-formed sponge material that was formed using methods other than mechanical shaping in addition to the mechanically-shaped sponge architecture. As such, a matrix material may have an alternation of empty and closed spaces, regular or irregular, having a defined geometry or amorphous, or a mixture of defined and irregular or amorphous spaces, in addition to the mechanically-shaped sponge architecture.

The shape and size of a sponge matrix material of the present disclosure can and will vary, and may depend on the end use of the sponge matrix material. For instance, a sponge matrix material for use in an industrial or environmental setting may be larger than a sponge matrix material for use in therapeutic situations.

The size of an absorbent matrix material may range from, for example, about 1 nm to about 25 mm, about 1 μm to about 100 μm, about 1 mm to about 1 meter, or more. Accordingly, the geometry of the matrix material may vary according to the intended use of the matrix material. Furthermore, different sizes and shapes of matrix material may be employed to selectively capture different particulate sizes and/or suspensions and/or colloids.

Further, the geometry of the matrix material may be selected to maximize packing or stacking efficiency, or maximize the number of members of the matrix material which may be packed and compressed into a device comprising more than one matrix material, thereby maximizing the absorption capacity of each device. The matrix material may be cylindrical, and may have an elliptical, oval, square, rectangular, triangular or polygonal or trapezoidal cross-section or shape. Further, various regular or irregular shapes or cross-sections may be used in forming the matrix material and devices comprising the matrix material, and a given member of a matrix material may comprise one or more discrete domains of a particular shape or cross-section. The geometries and cross-sections may support efficient packing, e.g., into devices, such that a sufficient number of members of matrix materials may be packaged in a device. The geometries, cross-sections, and configurations of any openings in the matrix material may further allow efficient absorption of fluids. Mixed geometries or shapes are also possible.

In some embodiments, the sponge matrix material may further comprise geometrically engineered structures able to collapse under mechanical, chemical or thermal action, or a combination thereof, and can subsequently assume an expanded shape. The expanded shape may be the original shape (before compression), or any other intended shape. In some embodiments, an absorbent matrix material has a size, shape and/or geometry configured for efficient packing into a small space, and/or configured to absorb and substantially retain material.

When the matrix material is compressible, the compressible matrix material may be compressed by subjecting to mechanical compression, thermal and/or chemical treatment (e.g., by chemically modifying the material, crosslinking, compounding or covalent bonding compounding, etc.). Size and/or volume of a compressible matrix material can be reduced in size to a size that is much smaller than the original, uncompressed size and/or expanded volume. For example, the compressed size or volume may be less than about 50% of the expanded size, or in other embodiments, less than about 40%, less than about 30%, less than about 25%, less than about 20%, less than about 15%, less than about 10%, less than about 5%, less than about 1%, less than about 0.5%, or less than about 0.1% of the expanded size or volume. Generally, after release of an applied mechanical compression or chemical constraint (e.g., by chemical reaction, breaking of cross-linking, etc.) or both, the material can assume its original, larger shape. A compressible matrix material of the present disclosure may be reduced in size and/or volume to a size and/or volume that is less than 50% of an expanded size of the matrix material.

The mechanism of expansion of the matrix material may also be elastic return, where the material, although not elastomeric per se, may still be stiff enough (e.g., the molecular creep time is sufficiently long) that the material may still at least partially assume its original shape after a period of compression and/or stress.

The compressible matrix material can be mechanically flattened and stacked, or simply compressed, then packaged in a device, e.g., in a conventional pharmaceutical capsule for ingestion. A compressible matrix material is generally made of an elastic and/or resilient material which can be compressed and is capable of absorbing liquid and/or material in the digestive tract. In some embodiments, one or more members of a compressible absorbent matrix material is packed into a device. For instance, the compressible property allows for efficient packing of a plurality of matrix material members into a small space, such as a capsule or other vehicle for storage or delivery.

A compressible matrix material can be made of any suitable material that can be used to form a compressible absorbent matrix material. Examples of suitable materials include: polymer or copolymer of polyurethane, nylon, polyethylene, polypropylene, polyacrylate, EVA, natural rubber, silicon, silicon rubber, latex, epdm rubber, butile rubber, nitrile rubber, PVA, PLA. Suitable elastomeric biomaterials include silicones, thermoplastic elastomers, polyolefin and polydiene elastomers, polyvinyl chloride, natural rubber, guayule rubber, heparinized polymers, hydrogels, polypeptides, and elastomers, which may be compounded with other polymer or natural or artificial elastomers and or fillers like clay, starch, elastic fibers, elastic microfibers, elastic nanofibers, which may be further compounded with inert and/or natural compounds, such as cellulose and its derivates, elastic and non-elastic fillers and powders.

Other suitable materials for a compressible matrix may include natural polymers such as cellulose, nanocellulose, bacterial cellulose, cellulose fibers, microfibers and nanofibers, methylcellulose, ethylcellulose, ethylmethylcellulose, other cellulose derivatives, cellulose compounded with natural or synthetic or artificial elastomers and/or fillers and natural hydrogels like chitosan, opuntia, and other disaccharides, and natural clays (e.g., montmorillonite).

The matrix material and devices comprising one or more members of the matrix material may further comprise other natural or synthetic materials to provide the desired mechanical properties, or desired absorptive and/or shielding properties. For instance, if absorbed fluid is intended to be protected from being subsequently released, the fluids should be kept inside or sequestered in the matrix material once absorbed. Accordingly, matrix material and devices comprising one or more members of the matrix material may further comprise natural or synthetic materials that facilitate gelling or increasing the viscosity of the absorbed fluid. Non-limiting examples of other materials may include hydrogel(s), fiber(s) and/or gum(s), and combinations thereof.

Other characteristics and compositions of matrix materials may be as described in, for example, U.S. Pat. No. 9,320,715 and US Patent Application No. 20140276330, the disclosures of which are incorporated herein in their entirety.

### II. Delivery Device

In an additional aspect, the present disclosure encompasses a delivery device comprising one or more members of a compressible absorbent matrix material. The matrix material has a mechanically-shaped sponge architecture. A compressible absorbent matrix material may be as described in Section I. A compressible absorbent matrix material may be prepared using a method described in Section III. Some characteristics of a device of the instant invention may be as described in Section I. Other characteristics and compositions of matrix materials may be as described in, for example, U.S. Pat. No. 9,320,715 and US Patent Application No. 20140276330, the disclosures of which are incorporated herein in their entirety.

### III. Method of Preparing

In another aspect, the present disclosure encompasses a method of preparing an absorbent matrix material having a mechanically-shaped sponge architecture. The method comprises providing a substrate and forming the sponge architecture in the substrate, forming a substrate around a removable porogenic growing matrix substantially having the shape of the sponge architecture and removing the porogenic growing matrix, or a combination thereof.

The substrate can and will vary and may be any substrate suitable for producing an absorbent matrix material. The substrate may be a naturally occurring material or may be an artificially prepared material. The substrate may have a defined geometry, an amorphous geometry, and combinations thereof.

The substrate may be a solid material or a hydrogel. The substrate may also be a sponge material. The sponge material may have open cells, closed cells or combinations of open and closed cells. The cells may have regular or irregular shapes, or a mixture of regular or irregular shapes.

The substrate may comprise elastomeric or resilient material. Elastomeric or resilient material may be used to prepare a compressible matrix material. Non-limiting examples of elastomeric or resilient material that may be employed for preparing a compressible matrix material include latex, guayule, polyurethane rubber, silicon rubber, cellulose, nanocellulose, nitrile rubber, or biological materials such as elastin, collagen and/mother natural proteins, or any other suitable materials that can retain and conserve their original shape after extended periods of time under compression, or which return to the memory shape or assume a different shape. Alternatively, the mechanism of expansion may be largely due to fluid absorption of the matrix material, for example, when including such materials as hemicellulose, which has a high absorption strength, and is able to create a hydraulic force to inflate the sponge scaffolding.

In some embodiments, the sponge matrix can be made of material safe for human ingestion. Non-limiting examples of suitable materials include: polymer or copolymer of polyurethane, nylon, polyethylene, polypropylene, polyacrylate, EVA, natural rubber, silicon, silicon rubber, latex, epdm rubber, butile rubber, nitrile rubber, PVA, PLA. Suitable elastomeric biomaterials include silicones, thermoplastic elastomers, polyolefin and polydiene elastomers, polyvinyl chloride, natural rubber, guayule rubber, heparinized polymers, hydrogels, polypeptides, and elastomers, which may be compounded with other polymer or natural or artificial elastomers and or fillers like clay, starch, elastic fibers, elastic microfibers, elastic nanofibers, which may be further compounded with inert and/or natural compounds, such as cellulose and its derivates, elastic and non-elastic fillers and powders.

Other suitable materials for the substrate include natural polymers such as carbohydrates, cellulose, nanocellulose, bacterial cellulose, cellulose fibers, microfibers and nanofibers, methylcellulose, ethylcellulose, ethylmethylcellulose, other cellulose derivatives, cellulose compounded with natural or synthetic or artificial elastomers and/or fillers and natural hydrogels like chitosan, opuntia, and other disaccharides, and natural clays (e.g., montmorillonite).

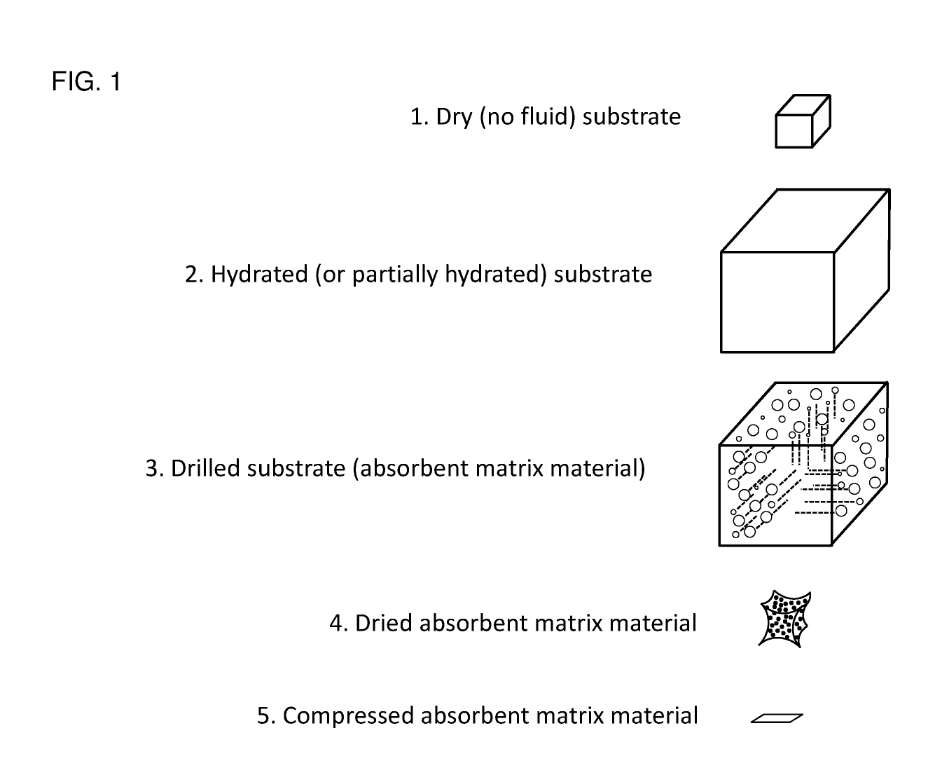

In some embodiments, a method comprises providing a substrate and forming the sponge architecture in the substrate (see e.g., FIG. 1). Non-limiting examples of forming the sponge architecture comprises drilling the substrate, puncturing the substrate, and combinations thereof. As used herein, the term “drilling” is used to refer to the removal of substrate material to produce cavities and generate a sponge architecture. Conversely, the term “puncturing” is used to refer to making invaginations or cavities without the removal of substrate material to produce a sponge architecture.

When the substrate material is a composition capable of adsorbing a fluid (referred to as hydration when the fluid is water; FIG. 1), the porous sponge architecture may be formed in a dry substrate, a substrate fully saturated with the fluid, or a substrate partially saturated with the fluid, or a combination thereof. This is beneficial, as the behavior of the resulting matrix material can be manipulated. For instance, if the porous sponge architecture is formed at 50% of the maximum potential hydration (fluid retaining) state of the substrate, then further hydration of the resulting matrix could shrink the drilled pores, which may be a useful characteristic in certain applications.

When the substrate is a dry hydratable substrate, a dry absorbent matrix material may be formed in the dry hydratable substrate. A dry matrix material may then be hydrated to generate a hydrated absorbent matrix material. Alternatively, the dry hydratable substrate may first be hydrated, and the porous sponge architecture may be formed in the hydrated substrate.

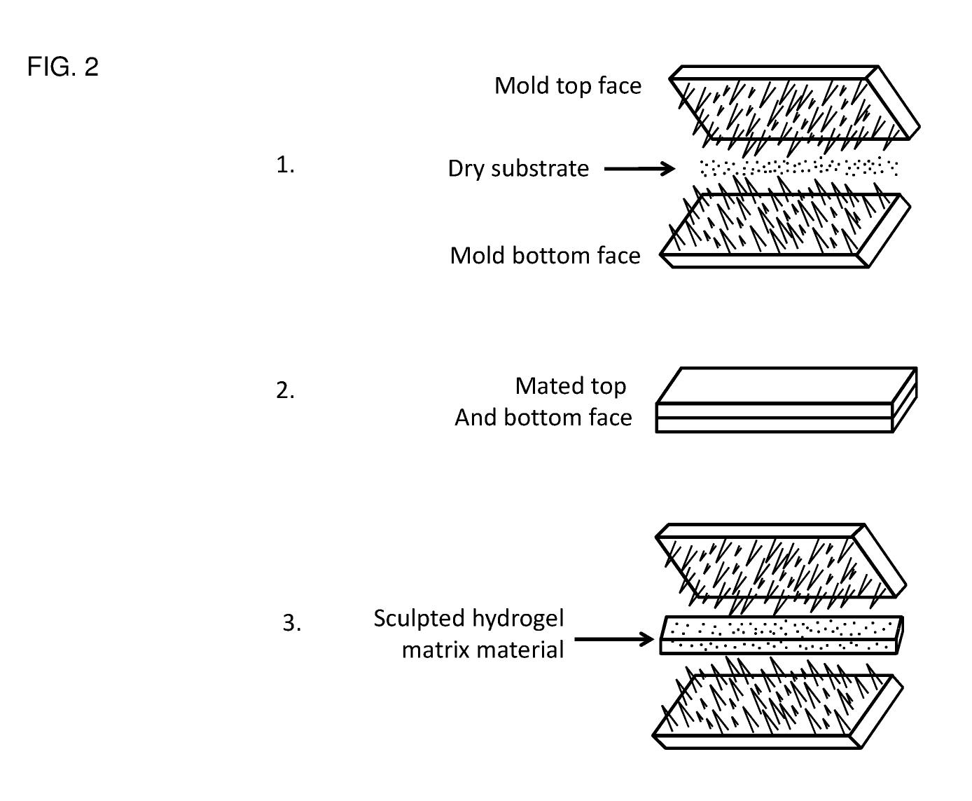

In other embodiments, a method comprises forming a substrate around a removable porogenic growing matrix (a mold) and removing the porogenic mold. In such embodiments, the porogenic mold has substantially the negative or opposite shape of the desired sponge architecture, thereby producing a mechanically-shaped sponge architecture substantially similar to the porogenic mold upon removal of the mold (FIG. 2).

Methods of forming a substrate around a removable mold are well known in the art and will vary depending on the substrate material, the removable mold, the desired sponge architecture, and combinations thereof. For instance, when a substrate is a dry hydratable carbohydrate, the dry carbohydrate may be deposited on, in, and/or around the removable mold, and hydrated to expand and form a hydrogel around the mold, thereby forming the absorbent matrix material. Alternatively, a substrate may be grown, built, deposited, or developed around the removable mold. Removing the growing matrix thus generates the matrix material having a mechanically-shaped sponge architecture substantially similar to the porogenic growing matrix.

The porogenic growing matrix may simply be removed by detaching the mold from the formed substrate thereby generating the porous matrix. Alternatively, the mold may be shape modified to allow detaching the porogenic growing matrix from the formed substrate. Further, the mold may be removed by eliminating the porogenic growing matrix from the formed substrate thereby preparing the porous matrix. For instance, the porogenic mold may be eliminated by chemically modifying the porogenic mold.

With either method, or importantly, the possible combinations of the two methods, the result is a chiseled, sculpted matrix material that could be dried (for many materials), compressed, stored, and manipulated. When the original shaped material is needed, it can be hydrated (or fluid bathed), and returns back to its original shape. This property is similar to a number of biological organisms, springing back to a functional biological living state from a dormant desiccated form.

### IV. Method of Using

In an additional aspect, the present disclosure encompasses methods of using compressible absorbent matrix material of the instant disclosure. Non-limiting examples of methods of using compressible absorbent matrix material include the use in a device for preventing absorption of nutrients or other compounds in the stomach to affect weight loss, the use for environmental clean-up, the use for stopping and preventing bleeding in a wound, or the use in imbibing blood or other fluids during surgery.

In one embodiment, compressible absorbent matrix material of the instant disclosure and/or devices comprising the matrix material may be used for affecting, among other things, weight loss and/or weight control, by sequestering nutrients or other compounds such as toxins from absorption in the digestive tract. See, for example, U.S. Pat. No. 9,320,715 and US Patent Application No. 20140276330, the disclosures of which are incorporated herein in their entirety.

### EXAMPLES

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Forming a Compressible Absorbent Matrix Material

A compressible absorbent matrix material may be prepared using a compressible hydrogel as the substrate (FIG. 1). The hydrogel may be provided in a dry state, and hydrated before forming the absorbent matrix in the hydrogel. The hydrogel may be mechanically formed by drilling of the substrate. Drilling may be by gouging out and removing hydrogel material to form pores, cells, and conduits and generate the sponge architecture in the hydrogel. The resulting matrix material may be dried then compressed to reduce its size.

### Example 2. Preparing an Absorbent Matrix Material by Forming a Substrate Around a Porogenic Mold

An absorbent matrix material may be prepared by forming a substrate around a removable porogenic growing matrix (a mold) and removing the porogenic mold (FIG. 2). The mold substantially has the shape of the desired sponge architecture. The porogenic mold may have a first face and a second face, wherein the first and second faces are matable. When mated, the first face and second face form a porogenic mold therebetween. A dry substrate material may be deposited in the porogenic mold and is hydrated to generate a sculpted hydrated absorbent matrix. The mold faces may then be removed to expose the absorbent matrix material.

### Example 2. Preparing an Absorbent Matrix Material by Building a Substrate Around a Porogenic Mold

An absorbent matrix material may be prepared by building a substrate around a removable porogenic mold and removing the porogenic mold. The porogenic mold has substantially the negative or opposite shape of the desired sponge architecture. A substrate material may be built in the spaces within the porogenic mold by depositing the substrate in and around the spaces within the mold. The mold may then be removed by eliminating the mold material from the formed substrate thereby preparing the porous device. The porogenic mold may be eliminated by chemically modifying the mold material.

What is claimed is:

1. A method of preparing an absorbent matrix material having a mechanically-shaped sponge architecture, wherein the method comprises:
a. providing a substrate and forming the sponge architecture in the substrate;
b. forming a substrate around a removable porogenic mold substantially having the shape of the sponge architecture and removing the porogenic mold; or
c. combinations thereof.

2. The method of claim 1, wherein the absorbent matrix material is compressible.

3. The method of claim 1, wherein the substrate is a naturally occurring material or an artificially prepared material.

4. The method of claim 1, wherein the substrate is a solid material, a hydrogel, or a sponge material.

5. The method of claim 1, wherein the method comprises forming the sponge architecture in a substrate.

6. The method of claim 5, wherein forming the sponge architecture is selected from the group consisting of drilling the substrate, puncturing the substrate, and combinations thereof.

7. The method of claim 5, wherein the substrate is selected from a hydrated substrate, a dry substrate, a partially hydrated substrate, or a combination thereof.

8. The method of claim 5, wherein the substrate is a dry hydratable carbohydrate.

9. The method of claim 5, wherein the substrate is a dry hydratable substrate and a dry absorbent matrix material is formed in the dry hydratable substrate.

10. The method of claim 9, wherein the method further comprises hydrating the dry absorbent matrix material to generate a hydrated absorbent matrix material.

11. The method of claim 5, wherein the substrate is a dry hydratable substrate and the method further comprises hydrating the substrate to generate a hydrated substrate, and forming the sponge architecture in the hydrated substrate.

12. The method of claim 1, wherein the method comprises forming a substrate around a removable porogenic mold substantially having the shape of the sponge architecture and removing the porogenic mold.

13. The method of claim 12, wherein the substrate is formed around the porogenic mold by growing, building, depositing, or developing the substrate around the porogenic mold.

14. The method of claim 12, wherein the substrate is selected from a hydrated substrate, a dry substrate, a partially hydrated substrate, or a combination thereof.

15. The method of claim 12, wherein the substrate is a dry hydratable carbohydrate.

16. The method of claim 12, wherein the substrate is a dry hydratable substrate, and the method further comprises depositing the dry hydratable substrate in the removable mold and hydrating the hydratable substrate to expand and form a hydrated substrate in the mold.

17. The method of claim 12, wherein the porogenic mold is removed by detaching the mold from the formed substrate.

18. The method of claim 12, wherein the porogenic mold is removed by shape modifying the mold to allow detaching from the formed substrate.

19. The method of claim 12, wherein the porogenic mold is removed by eliminating the mold from the formed substrate.

20. The method of claim 12, wherein the porogenic mold is removed by chemically modifying the porogenic mold to eliminate the mold.

21. The method of claim 2, wherein the matrix material has a defined size, and the method further comprises compressing the absorbent matrix material to reduce the size of the matrix material.

22. The method of claim 21, wherein the size of the matrix material is reduced to a size that is less than 50% of an expanded size of the matrix material.

23. The method of claim 1, wherein the absorbent matrix material is a hydrated absorbent matrix material, and the method further comprises drying the matrix material to form a dried absorbent matrix material.

24. The method of claim 2, wherein the absorbent matrix material is a hydrated absorbent matrix material having a defined size, and the method further comprises drying the matrix material to form a dried absorbent matrix material and compressing the dried matrix material to reduce the size of the dried matrix material.

25. A compressible absorbent matrix material having mechanically-shaped sponge architecture prepared using a method of claim 1.

26. The matrix material of claim 25, wherein the matrix material is reduced in size and/or volume to a size and/or volume that is less than 50% of an expanded size of the matrix material.

27. The matrix material of claim 25, wherein one or more member of the compressible absorbent matrix material is packed into a delivery device.

28. A delivery device comprising one or more members of a compressible absorbent matrix material, the matrix material having a mechanically-shaped sponge architecture prepared using a method of claim 1.
